(19) United States
(12) Patent Application Publication
(10) Pub. No.: US 2026/0041721 A1
(43) Pub. Date: Feb. 12, 2026

(54) Composition for Use Within a Vagina

(71) Applicant: Amelia Health Pty Ltd, McMahons Point (AU)

(72) Inventors: Catherine Jade Slogrove, McMahons Point (AU); Nada Millen, McMahons Point (AU)

(21) Appl. No.: 19/251,830

(22) Filed: Jun. 27, 2025

(30) Foreign Application Priority Data
Jun. 28, 2024 (AU) 2024901997

(51) Int. Cl. A61K 35/747; A61K 9/00; A61K 35/00; A61K 47/02; A61K 47/12; A61K 47/26; A61K 47/38; A61K 47/46

(52) U.S. Cl. CPC A61K 35/747; A61K 9/0034; A61K 47/02; A61K 47/12; A61K 47/26; A61K 47/38; A61K 47/46; A61K 2035/115

(57) ABSTRACT

A composition adapted for use within a vagina, the composition comprising: Lactobacillus crispatus and one probiotic selected from the group consisting of: Lactobacillus rhamnosus, Lactobacillus reuteri, and Lactobacillus gasseri.

### CROSS-REFERENCE

This is a U.S. non-provisional application claiming priority under 35 U.S.C. § 119(a) to Australian Application No. 2024901997, filed Jun. 28, 2024, the entire disclosure of which is incorporated herein by reference.

### TECHNICAL FIELD

The present invention relates to a composition for use within a vagina for supporting vaginal microbiome and/or for treatment of vaginal bacterial and yeast infections. More particularly, the combination of probiotic strains for vaginal route of application with a formulation to include prebiotics which are preserving viable probiotic organisms during shelf-life (in vitro) and nurture and support their growth at the moment of vaginal application (in vivo).

### BACKGROUND

Direct vaginal application showed higher cure rate compared to the same L. rhamnosus, GR-1™ and L. reuteri, RC-14™ strains administered orally (88% vs. 51%) in patients with bacterial vaginosis (BV). As shown in FIG. 1, the vaginal route of administration is faster in delivering the probiotic species to the site of action (FIG. 1 (right)). Also, by avoiding gastrointestinal environment from an oral application (FIG. 1 (left)), the full dose of probiotic organisms is delivered straight to the target, enabling the maximum beneficial effect. Being at the site of action, beneficial bacteria can directly replenish the vagina's environment by colonising it and producing beneficial substances like lactic acid that would further protect and support healthy vaginal microbiome. As shown in FIG. 1 (left), Oral probiotics need to go through the gastric system to get to the vagina. Being a harsh, acidic environment, many organisms may die before they reach the site of action. And while studies have shown oral probiotics do colonise in the vagina eventually, it can take longer than vaginal pessaries.

Vaginal administered probiotics are usually administered less frequently and for shorter periods of time (5-10 days). Orally administered probiotics usually need to be applied over course of 30 to 60 days.

Vaginal applicators, for example as disclosed in US Patent Publication No. 2018/0229016A1 have been developed for delivering a solid dosage form of a pharmaceutical preparation and vaginal applicators can be used to transvaginally deliver medicine to the apex of the vaginal canal. Disadvantages from using a vaginal applicator may be that there can be vaginal insertion injury when installing and/or mucosal irritation.

Any discussion of the prior art throughout the specification should in no way be considered as an admission that such prior art is widely known or forms part of common general knowledge in the field.

### SUMMARY

### Problems to be Solved

It may be an advantage to provide a composition for direct vaginal application than a composition for oral administration so that probiotics can be directly applied to the target region faster.

It may be an advantage to provide a dosage of Lactobacillus (L.) crispatus in the preferred colony forming units (CFU) range for providing an optimal beneficial effect to the patient/user.

It may be an advantage to provide a dosage of other active probiotics such as L. gasseri, L. rhamnosus, and L. reuteri in the preferred CFU range for providing a synergistic and beneficial effect to the patient/user when combined with the dosage of L. crispatus.

It may be an advantage to have the strains publicly available for obtaining the strains to prepare the formulation.

It may be an advantage to provide a plant based Vcap™ capsule for fast melting and delivering the active ingredients in the vaginal environment.

It is an object of the present invention to overcome or ameliorate at least one of the disadvantages of the prior art, or to provide a useful alternative.

### Means for Solving the Problem

A first aspect of the present invention may relate to a composition adapted for use within a vagina, the composition comprising: Lactobacillus crispatus and one probiotic selected from the group consisting of: Lactobacillus rhamnosus, Lactobacillus reuteri, and Lactobacillus gasseri.

Preferably, the composition comprises Lactobacillus crispatus and two probiotics selected from the group.

More preferably, the composition comprises Lactobacillus crispatus and three probiotics selected from the group.

Preferably, Lactobacillus crispatus is Lactobacillus crispatus LCr86.

Preferably, Lactobacillus rhamnosus is Lactobacillus rhamnosus, GR-1™; wherein Lactobacillus reuteri is Lactobacillus reuteri, RC-14™; and wherein Lactobacillus gasseri is Lactobacillus gasseri UALg-05.

Preferably, the composition is in the form of a hardshell capsule.

Preferably, the composition is in the form of a gel.

Preferably, the composition is in the form of a tablet.

Preferably, the composition further comprises prebiotic excipients comprising: skim milk.

Preferably, the composition further comprises prebiotic excipients comprising: skim milk, and maltose.

Preferably, the amount of maltose is less than 0.01 grams.

Preferably, the composition further comprise: microcrystalline cellulose, colloidal anhydrous silica, and magnesium stearate.

Preferably, the composition comprising between 1×10⁹ to 2×10⁹ CFU Lactobacillus crispatus per gram.

Preferably, the composition comprising between 1×10⁹ to 2×10⁹ CFU Lactobacillus gasseri per gram.

Preferably, the composition comprising between 1.00×10⁹ to 2.5×10⁹ CFU Lactobacillus rhamnosus per gram.

Preferably, the composition comprising between 1.00×10⁹ to 2.5×10⁹ CFU Lactobacillus reuteri per gram.

Preferably, a method of supporting vaginal microbiome using the composition, the method comprising the step of vaginally applying the composition once daily.

Preferably, a method for treatment of vaginal bacterial and yeast infections using the composition, the method comprising the step of vaginally applying the composition once or twice weekly for six months.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1 illustrates the differences in oral application and vaginal application.

FIG. 2A illustrates a first patient's Vaginal Microbiome Profile before the application of L. crispatus LCr86 for 7 days.

FIG. 2B illustrates the first patient's Vaginal Microbiome Profile after the application of L. crispatus LCr86 for 7 days.

FIG. 3A illustrates a second patient's Vaginal Microbiome Profile before the application of L. crispatus LCr86 for 7 days.

FIG. 3B illustrates the second patient's Vaginal Microbiome Profile after the application of L. crispatus LCr86 for 7 days.

FIG. 4 illustrates a graph showing a similar strain of L. crispatus CTV-05 used for demonstrating that participants with already good colonization of L. crispatus have colonization rate of 30% versus participants with depleted L. crispatus colonies that had colonization rate of over 80%.

FIG. 5 illustrates charts relating to the vaginal microbiome of reproductive-age women according to ethnicity.

FIG. 6A is a chart illustrating cell counts and viability at room temperature or at 4° C. monitored over a predetermined period of time based on using water as a control.

FIG. 6B is a chart illustrating cell counts and viability at room temperature or at 4° C. monitored over a predetermined period of time based on using skimmed milk as an expedient.

FIG. 6C is a chart illustrating cell counts and viability at room temperature or at 4° C. monitored over a predetermined period of time based on using inulin as an expedient.

FIG. 6D is a chart illustrating cell counts and viability at room temperature or at 4° C. monitored over a predetermined period of time based on using maltodextrin as an expedient.

FIG. 6E is a chart illustrating cell counts and viability at room temperature or at 4° C. monitored over a predetermined period of time based on using sucrose as an expedient.

### DESCRIPTION OF THE INVENTION

Preferred embodiments of the invention will now be described with reference to the accompanying drawings and non-limiting examples.

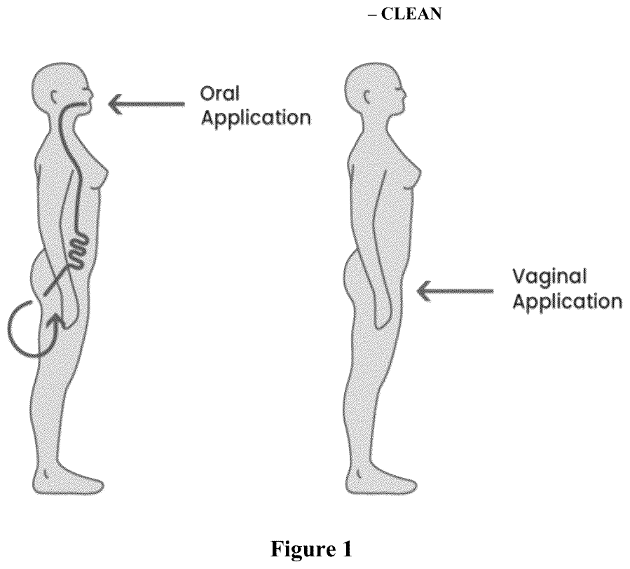

In an embodiment of the present invention, as illustrated in FIG. 1. FIG. 1 shows a person that is using an oral application of a composition (left) compared to a person that is using a vaginal application of a composition (right).

In an embodiment of the present invention, there may be a composition for use within a vagina, the composition comprising: Lactobacillus crispatus and one probiotic selected from the group consisting of: Lactobacillus rhamnosus, Lactobacillus reuteri, and Lactobacillus gasseri. As too much lactobacilli in the vagina can disadvantageously cause the condition of cytolytic vaginosis, in which the condition results in lysis of vaginal epithelial cells, there is a long-felt need for delicate balancing the number of lactobacilli in the composition that allows for the synergistic benefit in the vaginal environment between lactobacilli species while going below the amount threshold for avoiding cytolytic vaginosis.

Group studies conducted on healthy women measured improvement in vaginal microbiome upon dosage for 5-10 days of Lactobacillus rhamnosus, GR-1™ and Lactobacillus reuteri, RC-14™ in concentrations of:

Group 1: a dosage of 8.0×10⁸ CFU of each strain per day;
Group 2: two dosages of 8.0×10⁸ CFU of each strain per day;
Group 3: a dosage of 6.0×10⁹ CFU each strain per day;
Group 4: a dosage of Lactobacillus rhamnosus, GG of 1.0×10¹⁰ CFU per day.

From the group studies, it was discovered that higher doses than 8.0×10⁸ CFU is needed to make an improvement in vaginal microbiome. And that the Group 2 had the best and statistically significant results, and that higher doses per day in Group 3 such as 6×10⁹ CFU and Group 4 such as 1.0×10¹⁰ CFU did not demonstrate significant improvement but rather rendered a healthy microbiome unstable. As such, the dosage and number of lactobacilli used in the formulation or composition was optimised from the result of the studies, in which the statistically significant improvement in vaginal health was achieved using 2.5×10⁹ CFU of Lactobacillus rhamnosus, GR-1™ and Lactobacillus reuteri, RC-14™ mixture. In similar studies conducted and interpolation of the trend, for Lactobacillus crispatus and Lactobacillus gasseri, the dose of 2.0×10⁹ CFU was chosen.

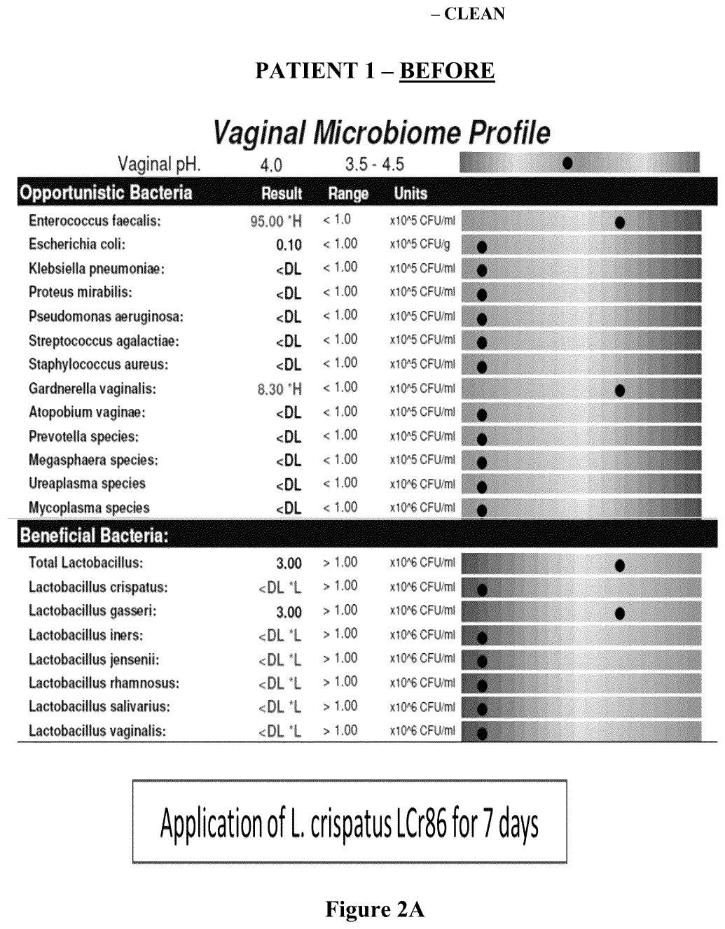

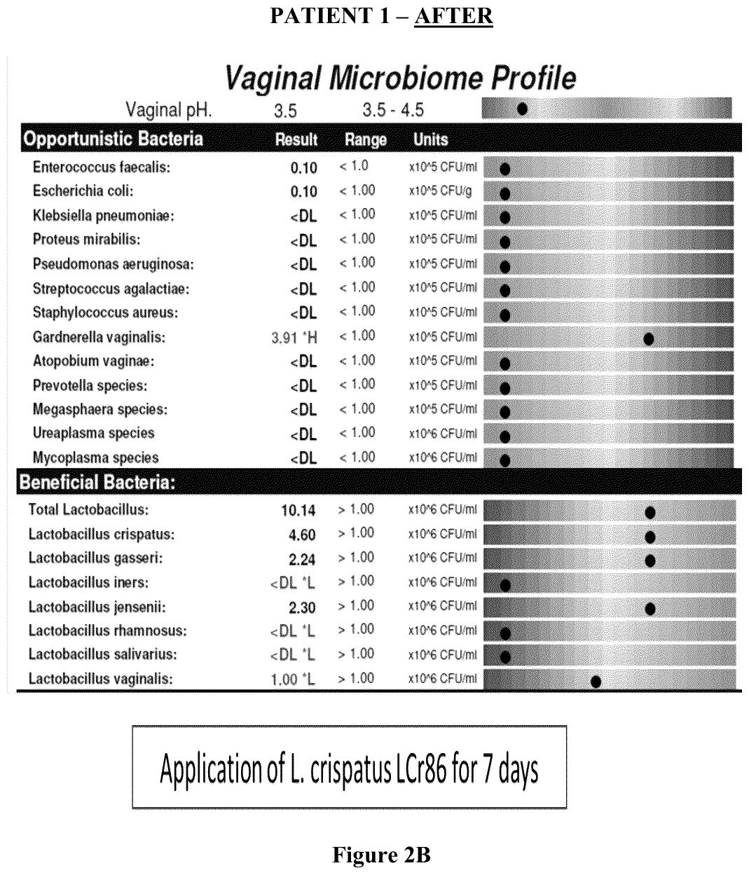

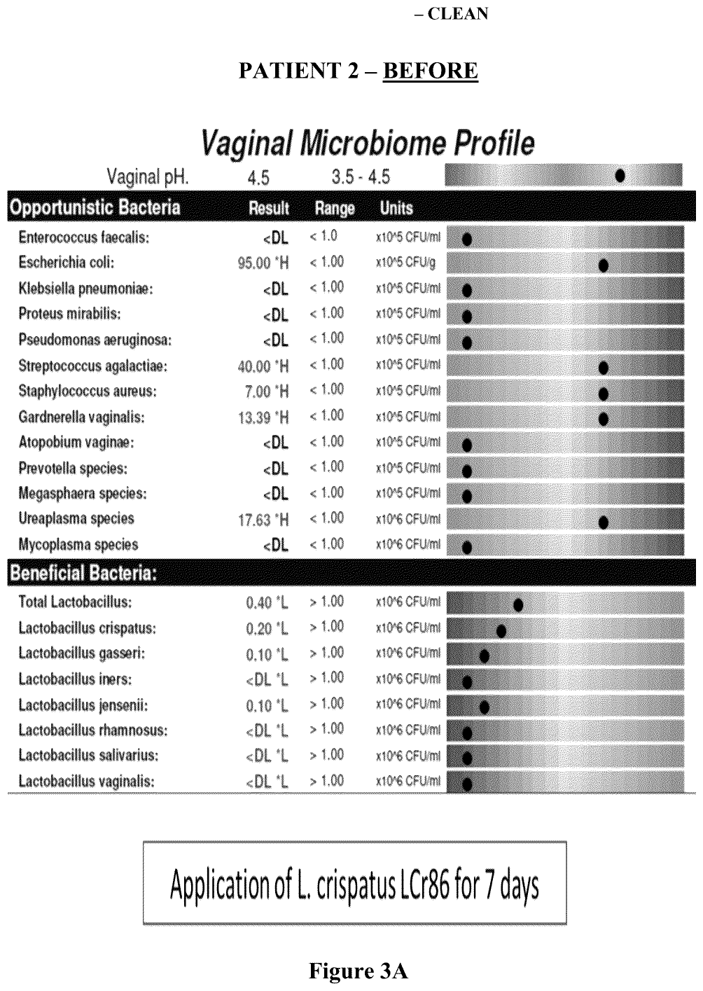

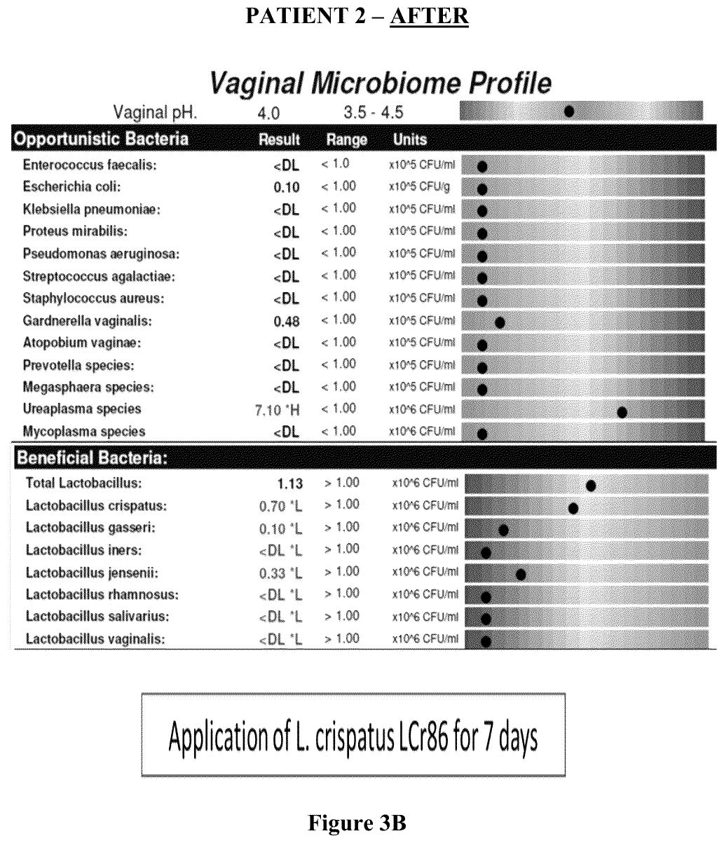

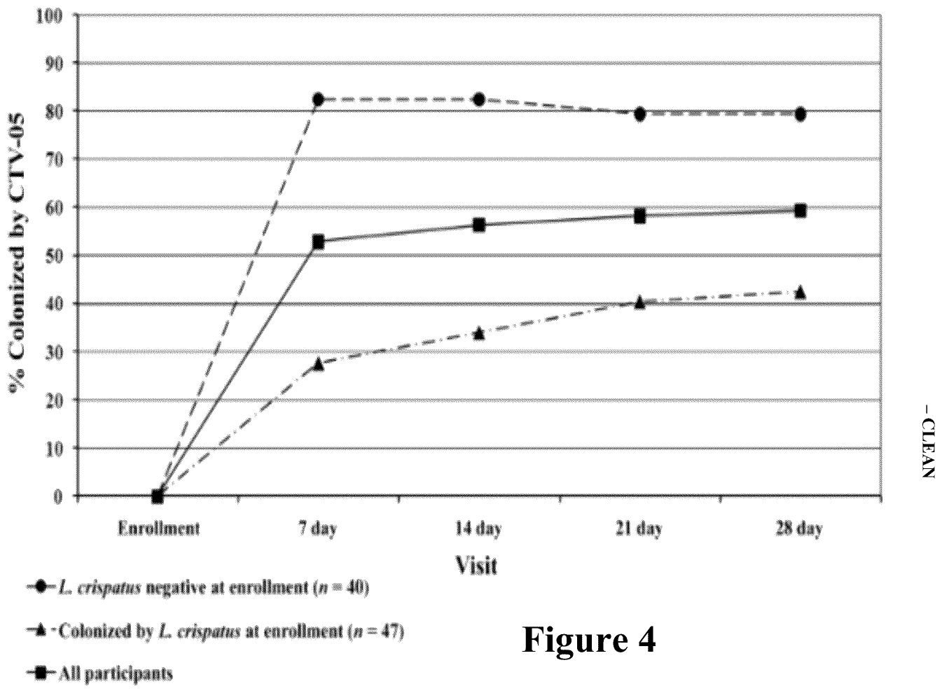

From the studies, it was discovered that the concentration of 6.0×10⁹ CFU is too much which does not improve vaginal microbiome health and making it more unstable. This is because essentially, adding a lot of microorganisms to a limited space and food supply (from the excipients and glycogen produced by vaginal epithelial cells) will not further improve nor make things better. Extra abundant probiotic organisms will only fight for food and colonizing space more intensely and enter into a competition with each other. Once organisms reach to a certain concentration, further addition of more of the same organisms will not result in a successful colonization. As such, a microbiome balance is needed. As shown in FIGS. 2A-3B, study participants with already good colonization of Lactobacillus crispatus have a colonization rate of 30% compared to participants with depleted Lactobacillus crispatus colonies that had colonization rate of over 80%. While the study is using a similar strain of Lactobacillus crispatus, such as Lactobacillus crispatus CTV-05 as shown in FIG. 4, the teachings and principle of using the similar strain can be of a similar expectation to using Lactobacillus crispatus LCr86 strain.

Furthermore, too much of lactobacilli can cause the condition called cytolytic vaginosis. And this condition is characterized by an abundant growth of lactobacilli resulting in lysis of vaginal epithelial cells. As such, through the group studies, the optimal range of lactobacilli in the formulation would not cause cytolytic vaginosis.

The benefit of vaginal application versus oral application is shown in FIG. 4. Direct vaginal application showed higher cure rate compared to the same L. rhamnosus, GR-1™ and L. reuteri, RC-14™ strains administered orally (88% vs. 51%) in patients with bacterial vaginosis. The vaginal route of administration is faster in delivering the probiotic species to the site of action. Also, by avoiding gastrointestinal environment, the full dose of probiotic organisms is delivered straight to the target, enabling the maximum beneficial effect. Being at the site of action, beneficial bacteria can directly replenish the vagina's environment by colonising it and producing beneficial substances like lactic acid that would further protect and support healthy vaginal microbiome. Oral probiotics need to go through the gastric system to get to the vagina. Being a harsh, acidic environment, many bacteria may die before they reach the site of action. And while studies have shown oral probiotics do colonise in the vagina eventually, it can take longer than vaginal pessaries. Vaginal administered probiotics are usually administered less frequently and for shorter periods of time (5-10 days). Orally administered probiotics usually need to be applied over course of 30 to 60 days.

Preferably, the composition may comprise 2.0×10⁹ CFU of Lactobacillus crispatus, and 2.0×10⁹ CFU of Lactobacillus gasseri. More preferably, besides Lactobacillus crispatus and Lactobacillus gasseri, the composition further comprises 1.25×10⁹ CFU of Lactobacillus rhamnosus, and/or 1.25×10⁹ CFU of Lactobacillus reuteri. As probiotics requires food supply, it is preferable for the composition to further comprise excipients such as a non-fat dry milk powder or skim milk. The composition may further comprise microcrystalline cellulose, colloidal anhydrous silica, and magnesium stearate. It may also be beneficial for the composition to further comprise another excipient such as malt sugar or maltose. As all the components of the composition requires to be stored, a hardshell capsule or more preferably, a plant based Vcaps™ plus capsule is used.

An advantage of using Vcaps™ plus capsule is that the capsule dissolves quickly and predicably at different pH values and the capsule dissolves with organisms and other food components such as the expedients. Further, this type of capsule offers additional advantages such as a lower moisture content and that the capsules are inert and are robust against extreme storage conditions. Further, this type of capsule has a similar performance of gelatin, in which gelatin-like dissolution advantageously eliminates the use of gelling agents which if used, may slow down release of active ingredients. A common problem from prolonged usage of capsules is that it creates a drying problem to the vagina. The solution of using skim milk is that it can also act as a moisturising or soothing element when released from the capsule in the vaginal environment.

Further the benefit of using the hardshell capsule over a gel is that the ease of administration or insertion is better with a capsule than with gel. Also, insertion of a capsule does not require vaginal applicator, which improves insertion safety. With capsule, there is less and minimal vaginal discharge that is following administration. The hardshell capsule is not perceived as sticky. Additionally, the product is odorless and does not prevent the patients from their daily activities such as work or sport etc., as it is applied before bed time. This improves efficacy, safety and patient's compliance.

Further, the benefit of using the hardshell capsule over tablets is that the capsule formulation or composition does not require vaginal secretions to activate the formulation.

Tablets require vaginal secretions to disintegrate and active agents in a tablet can be released in up to 30 minutes, whereas plant based hardshell capsule melts in the vaginal environment within 5-10 minutes, which allows the full active combination of probiotics to be released to the vaginal environment sooner compared to a tablet form of application.

As the composition combines the builder and fighter Lactobacillus strains, the combination of strains provides synergistic effect on vaginal microbiome, in which the combination of strains acts to restore beneficial species colonies, inhibit overgrowth of pathogenic bacteria, and also reduce the occurrence of recurring infections.

Lactobacillus crispatus is the most dominant Lactobacillus strain in a vagina and the most important beneficial vaginal strain present in vaginal microbiome of approximately 65% women across different ethnic groups, in which the Lactobacillus species are shown in the pie charts in FIG. 5. Its dominance advantageously indicates that there is a balanced and healthy vaginal microbiome and Lactobacillus crispatus creates very stable colonies which protect vaginal environment from pathogen species that commonly live in the vagina to overgrow and that Lactobacillus crispatus colonized in vagina of women have a fivefold decreased risk for developing bacterial vaginosis (BV). It is an advantage to include in the composition Lactobacillus gasseri together with Lactobacillus crispatus as Lactobacillus gasseri dominates in vaginal microbiome of 5-8% of women and as such rarely transitions toward bacterial vaginosis (BV) state. Lactobacillus gasseri beneficially attacks pathogens by releasing antimicrobial substances within the vagina and interfere with the pathogen's adherence and biofilm formation there. Further, the Lactobacillus species produce lactic acid, which lowers the pH and making the vaginal environment acidic, in which most pathogenic bacteria are inhibited and will not survive in low pH environment.

While the charts in FIG. 5 show that Lactobacillus iners is the second most common bacteria in a healthy vagina, unlike with Lactobacillus crispatus, Lactobacillus iners is associated with both normal and abnormal microflora. As Lactobacillus iners are described as a ‘transitional’ species, when Lactobacillus iners has increased in a higher ratio than other “good” bacteria, it may indicate that the vaginal microbiome is on the way to become disrupted and imbalanced. As Lactobacillus iners is less protective against pathogenic bacteria, women colonized dominantly with Lactobacillus iners have greater risk for developing bacterial vaginosis (BV) compared with women colonized predominantly with Lactobacillus crispatus.

While the charts in FIG. 5 show that Lactobacillus jensenii is another frequently detected good bacteria in a healthy vagina, its representation in the vaginal population varies in different ethnic groups. As currently Lactobacillus jensenii is not on the list of permitted ingredients for use in medical products in Australia, it may be on the list of permitted ingredients in the future and similar numbers of CFUs can form part of an improved composition should the Australian or other overseas jurisdictions allow.

The use of universal “builder” species makes the composition applicable for restoring vaginal microbiome across different ethnic groups. This composition and product provide probiotic species that are present in healthy vaginal microbiome of 40-65% women universally.

Fighter strains present in the formulation is desirable. Though beneficial strains above have ability to push back the pathogen species (by producing lactic acid, hydrogen peroxide and other antimicrobial substances), L. rhamnosus, GR-1™ together with L. reuteri, RC-14™ have been extensively studied for its beneficial effect in restoring healthy vaginal microbiome in patients with bacterial vaginosis and vaginal yeast infection. It was shown that these strains produce antimicrobial substances that disrupt pathogen's growth (by direct antimicrobial effect, by preventing their adhesion the vaginal wall, or disrupt their pathogens'biofilms. Clinical studies have found that these two strains are more effective than placebo, and as equally effective as antibiotic treatments in restoring healthy vaginal microbiome. Also, a treatment with antibiotics and antifungals is more effective if used in combination with these probiotic strains.

These strains also are able to reduce unpleasant symptoms that follow vaginal infections, such as irritation, itch, and discharge.

The benefit of these strains can be also seen through reduction in vaginal infection recurrent episodes. Studies have shown that recurrency rates of BV are at 15-30% within the first 3 months and at more than 50% within a year after the antibiotic treatment oral metronidazole. BV relapses occurred for around 44% of the patients followed up 24 months even after more aggressive initial antibiotic treatment. Yeast infections will affect 75% of women at some point in their life. Half of the women that get pharmaceutical treatment for yeast infections will experience the infection again, and 9% globally will experience the condition recurrently (more than 4 episodes a year). That's nearly one in 10 women. Randomised controlled trials over a period of more than 20 years showed that the use of Lactobacillus rhamnosus, GR-1™ and Lactobacillus reuteri, RC-14™ at a dose of at least 10⁸ CFU/day for 2 months normalize the vaginal flora, help cure the existing infection and prevent recurrence of BV. The use of probiotics in encouraged for long term control of BV relapses. Lactobacillus rhamnosus, GR-1™ and Lactobacillus reuteri, RC-14™ are obtained as Urex™ probiotic blend from CHR HANSEN (https://www.chr-hansen.com/en/human-health-and-probiotics/our-probiotic-strains/urex).

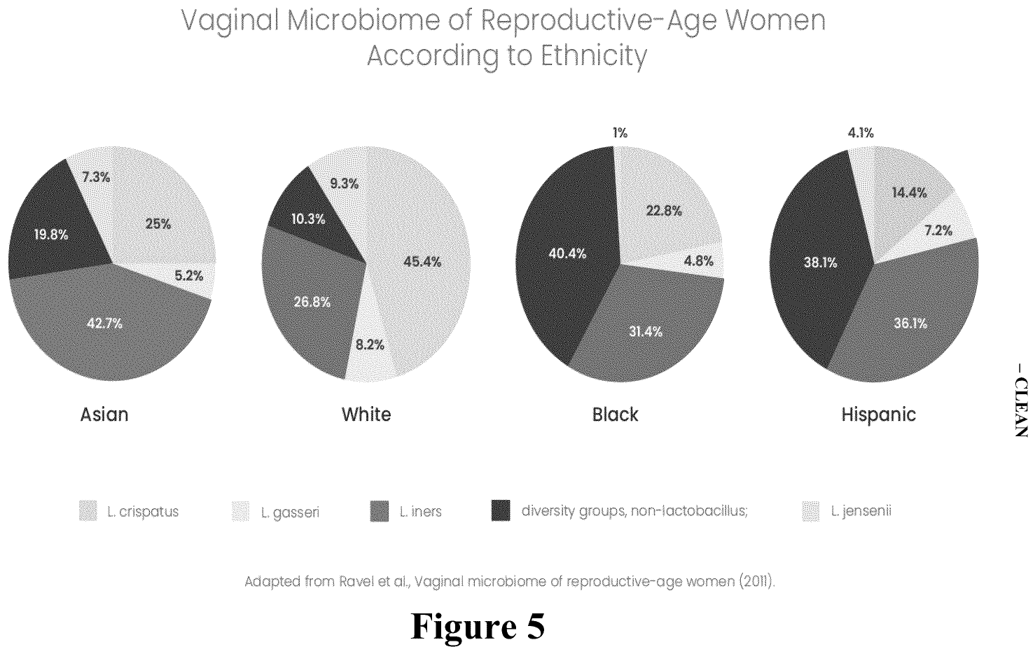

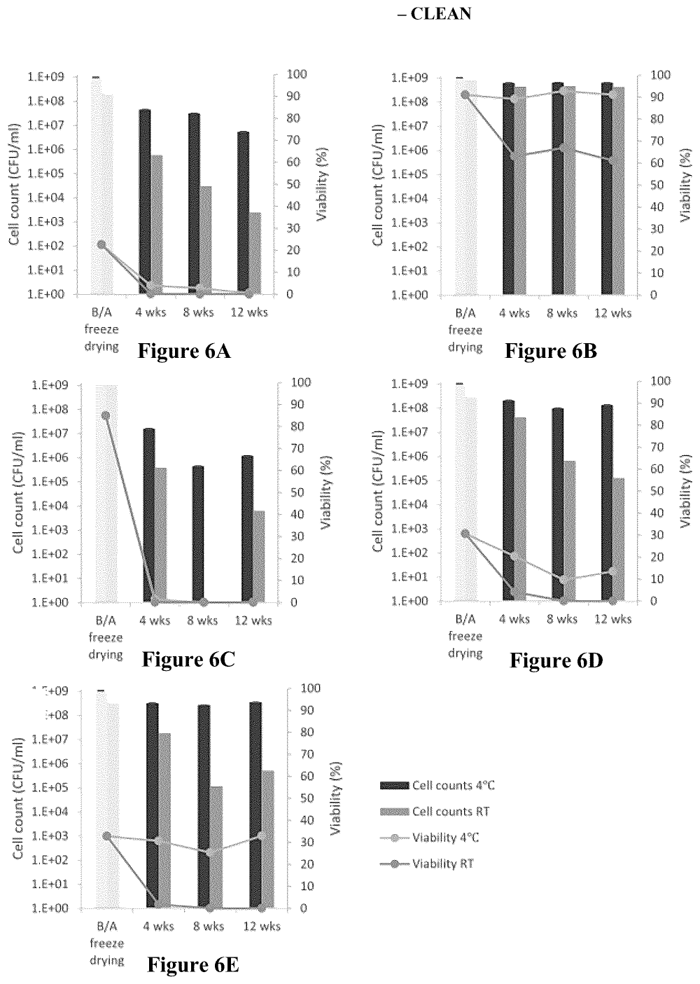

The benefit of choosing skim milk or non-fat milk powder as the expedient compared to other expedients is shown in FIGS. 6A to 6E, more particularly the chart as illustrated in FIG. 6B. The formulation has been designed with the protective and nurturing effect on probiotic organisms in mind. Excipients have been carefully chosen to ensure viability of sensitive dried lactobacilli bacteria, protecting them from moisture and degradation. These excipients are also gentle on vaginal wall tissue and thus support product targeted action. Further advantage of skim milk was shown to have highly protective effect on probiotic species in preserving them over shelf-life period at room temperature. Skim milk is also found to be superior to other commonly used cryoprotectants, such as inulin, as shown in FIG. 6C; maltodextrin, as shown in FIG. 6D; and sucrose, as shown in FIG. 6E.

Besides keeping the formulation stable and probiotic powder safe from water activity, the excipients are carefully chosen to play a prebiotic role. Therefore, a prebiotic must be digestible by probiotic organisms in the formulation, but by pathogen bacteria and yeast. Lactose from skim milk is considered a prebiotic. It can be digested by L. crispatus and easily digested by L. rhamnosus, GR-1™. Finally, skim milk contains and adsorbs less water than other commonly used prebiotics, making the probiotic organisms more stable and safer during shelf life.

Other commonly used prebiotics and the reasons why those ingredients are not included in this formulation are listed below. The selection of prebiotics was based on the ability to preserve viability of probiotic organisms. The most important properties of potential elected prebiotics were their: a) digestibility by probiotic organisms; b) water content and activity; c) hygroscopicity (ability to attract water); and d) regulatory clearance by TGA.

Excipients considered but not passing all the above criteria a) to d) are:

GOS which is not allowed for use by TGA in this category;
FOS which is digestible by Candida species, and it can worsen yeast vaginal infection;
Maltose which is digestible by Candida species, and it can worsen yeast vaginal infection (reduced to minimal concentration in the formulation);
Glucomannan which is hygroscopic;
Lactulose which is hygroscopic with high water content;
Inulin which is hygroscopic with high water content, and increases pathogen bacteria concentration;
Trehalose which is high acidity, has high water content, and restricted to oral use;
Maltodextrin which has high water content, and can increase pathogen bacteria concentration;
D-galactose which is not allowed for use by TGA in this category;
Fructose which is hygroscopic;
Mannitol which is hygroscopic.

Studies and clinical benefit of L. crispatus LCr86 applied once a day for 7 days, in which the formulation contains skim milk or skim milk and maltose, is that it has improved pH value, reduced pathogen species, and increased numbers of beneficial lactobacilli species.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

The invention claimed is:

1. A composition adapted for use within a vagina, the composition comprising:
Lactobacillus crispatus and one probiotic selected from the group consisting of:
Lactobacillus rhamnosus, Lactobacillus reuteri, and Lactobacillus gasseri.

2. The composition according to claim 1, wherein the composition comprises Lactobacillus crispatus and two probiotics selected from the group.

3. The composition according to claim 2, wherein the composition comprises Lactobacillus crispatus and three probiotics selected from the group.

4. The composition according to claim 3, wherein Lactobacillus crispatus is Lactobacillus crispatus LCr86.

5. The composition according to claim 4, wherein Lactobacillus rhamnosus is Lactobacillus rhamnosus, GR-1™; wherein Lactobacillus reuteri is Lactobacillus reuteri, RC-14™; and wherein Lactobacillus gasseri is Lactobacillus gasseri UALg-05.

6. The composition according to claim 5, wherein the composition is in the form of a hardshell capsule.

7. The composition according to claim 5, wherein the composition is in the form of a gel.

8. The composition according to claim 5, wherein the composition is in the form of a tablet.

9. The composition according to claim 5, wherein the composition further comprises prebiotic excipients comprising skim milk.

10. The composition according to claim 5, wherein the composition further comprises:
skim milk, and maltose.

11. The composition according to claim 10, wherein the amount of maltose is less than 0.01 grams.

12. The composition according to claim 11, wherein the composition further comprise:
microcrystalline cellulose, colloidal anhydrous silica, and magnesium stearate.

13. The composition according to claim 12, comprising between 1×10⁹ to 2×10⁹ CFU Lactobacillus crispatus per gram.

14. The composition according to claim 13, comprising between 1×10⁹ to 2×10⁹ CFU Lactobacillus gasseri per gram.

15. The composition according to claim 14, comprising between 1.00'10⁹ to 2.5×10⁹ CFU Lactobacillus rhamnosus per gram.

16. The composition according to claim 15, comprising between 1.00×10⁹ to 2.5×10⁹ CFU Lactobacillus reuteri per gram.

17. A method of supporting vaginal microbiome using the composition according to claim 16, the method comprising the step of vaginally applying the composition once daily.

18. A method for treatment of vaginal bacterial and yeast infections using the composition according to claim 16, the method comprising the step of vaginally applying the composition once or twice weekly for six months.
